(19) United States
(12) Patent Application Publication
(10) Pub. No.: US 2025/0362209 A1
(43) Pub. Date: Nov. 27, 2025

(54) Air Collection and Storage System and Method for Volatile Organic Compound Analysis with Improved Integrity and Reproducibility

(71) Applicant: Guillermo Vidal de MIguel, Madrid (ES)

(72) Inventor: Guillermo Vidal-de-Miguel, Madrid (ES)

(73) Assignee: Guillermo Vidal-de-Miguel, Madrid (ES)

(21) Appl. No.: 19/211,615

(22) Filed: May 19, 2025

Related U.S. Application Data
(60) Provisional application No. 63/651,483, filed on May 24, 2024.

(51) Int. Cl. G01N 1/22; G01N 33/497

(52) U.S. Cl. CPC G01N 1/22; G01N 33/4975; G01N 2001/2244

(57) ABSTRACT

A breath sampling and preservation system is disclosed. The system comprises an air storage unit that includes a chemically inert film bag enclosed in a rigid, thermally insulated canister. The canister includes an inlet valve, a sample port, and a secondary port allowing pressure to be applied to the exterior of the film bag, which is used for storing a portion of a breath sample containing airborne analytes such as volatile organic compounds (VOCs), gases, vapors, or aerosols. The sample is typically collected using a breath inlet and sensor system that selects a predefined fraction of the exhalation based on real-time analysis of parameters such as CO₂ or flow. After the fraction of interest is introduced into the bag, the inlet valve is closed, and the air storage unit is placed in a temperature-controlled preservation bed until analysis.

### CROSS REFERENCE TO RELATED APPLICATION

This application claims the benefit of U.S. Provisional Application No. 63/651,483, filed May 24, 2024, which is hereby incorporated by reference in its entirety

### FIELD OF THE INVENTION

. The present invention relates to air storage systems, specifically to devices designed for the collection, preservation, and analysis of air samples, particularly those containing volatile organic compounds (VOCs). These systems are crucial in environmental monitoring, medical diagnostics, and various industrial applications where precise air sample analysis is required.

### BACKGROUND OF THE INVENTION

Current air sampling and storage methods face several challenges, particularly in maintaining the integrity of the samples over extended periods and during transportation. Traditional sampling bags, such as those made from polyethylene terephthalate (PET), are known to retain low volatility molecules well but only for short durations due to their permeability and susceptibility to contamination.

The collection and preservation of air samples for analysis is of significant interest in various fields, including environmental monitoring, medical diagnostics, industrial safety, and scientific research. Air sample analysis is critical for detecting and quantifying volatile organic compounds (VOCs), pollutants, biomarkers, and other airborne substances that can have profound impacts on human health, environmental quality, and industrial processes.

The advancement of air sampling technologies, particularly those that offer improved preservation and integrity of samples, addresses critical challenges in these fields. Innovations that prevent contamination, reduce sample degradation, and maintain consistent handling conditions are particularly valuable, enhancing the reliability and accuracy of air sample analysis.

Breath analysis has emerged as a promising diagnostic tool due to its non-invasive nature and the wealth of information it can provide about a person's health. The breath contains various volatile organic compounds (VOCs) and other molecules that are byproducts of metabolic processes. The concentration of these molecules can offer insights into physiological and pathological states, making breath analysis a valuable technique for early disease detection, monitoring of disease progression, and assessment of treatment efficacy. Detecting these metabolites comes with specific requirements:
(i) Sensitivity and Precision: The diagnostic potential of breath analysis relies on the ability to detect and measure the concentration of biologically relevant molecules accurately. These molecules often occur at minute concentrations (parts-per-trillion levels), requiring highly sensitive and precise instruments.
(ii) Sample Integrity: The concentration of molecules in breath carries critical information. Therefore, it is essential to preserve the integrity of the breath samples from the moment of collection through to analysis. This involves preventing contamination, and avoiding condensation and diffusive losses of VOCs to ensure that the sample composition remains unchanged.
(iii) High-Performance Instruments: Advanced analytical techniques and instruments are necessary to meet the stringent requirements of breath analysis. One such high-performance instrument is the Secondary Electrospray Ionization-High Resolution Mass Spectrometry (SESI-HRMS). SESI-HRMS combines the sensitivity of secondary electrospray ionization with the high resolution and accuracy of mass spectrometry, allowing for the detection and quantification of a wide range of VOCs and other compounds in breath samples.

One option for breath sample collection for offline analysis is the use of thermal desorption (TD) tubes. These tubes offer logistical advantages, such as being compact, lightweight, and easy to transport. However, they also present significant drawbacks:
(i) Adsorption-Desorption Process: The process of adsorbing VOCs onto the TD tube and subsequently desorbing them for analysis can be slow and has the potential to introduce artifacts, compromising the accuracy of the analysis. The efficiency of desorption can vary, leading to incomplete recovery of the analytes.
(ii) Tube-to-Tube Reproducibility: Ensuring consistent performance across multiple TD tubes can be challenging. Variations in tube manufacturing, conditioning, and handling can result in differences in adsorption and desorption efficiencies, impacting the reliability of results.

For biomarker discovery, where detecting subtle changes in VOC concentrations is critical, the limitations of TD tubes become more pronounced. The slow process and the potential for introducing artifacts adsorption-desorption tube-to-tube reproducibility can obscure true biological signals and complicate the identification and quantification of biomarkers, making it difficult to draw accurate conclusions.

Direct collection of air samples in canisters is another method used for air collection for analysis. The use of canisters for air sample collection is well-documented in the EPA TO-015 method. In this approach, the canister is prepared by applying vacuum to it. To collect a sample, the valve of the canister is opened, causing the vacuum of the canister to pulls the sample into it. Canisters are highly impermeable, and their surface can be functionalized with coatings to enhance sample preservation. However, the use of canisters presents several significant disadvantages:
(i) Vacuum sampling: Using a vacuum to collect breath samples can be potentially harmful to the individual providing the sample. The process of creating a vacuum can cause discomfort or distress, particularly for individuals with respiratory conditions. The rapid change in pressure can also lead to inadvertent inhalation of contaminants or other particles if the equipment is not perfectly clean. This risk further complicates the use of canisters for breath analysis.
(ii) Condensation of Large Molecules: As the gas expands and cools under the low pressure inside the canister, large molecules tend to condense. This condensation can alter the composition of the sample, leading to inaccurate analysis results. The cooling effect associated with the vacuum process makes it difficult to maintain large VOCs in their gaseous state, compromising the integrity of the sample. Although canisters are highly impermeable, the expansion and cooling of the sample can still lead to degradation of certain compounds. This is especially challenging for the detection of biomarkers in breath samples, where precise quantification of various compounds is necessary.

Sampling bags are a widely used option for breath analysis due to their ability to be inflated with very low pressure drops, which is a key advantage in this application. A typical configuration includes a bag and a valve, and these bags come in various shapes and materials. Tedlar, Kynar, and Aclar films are known for their inertness and low permeability. However, several studies suggest that PET (Nalophan) film shows the best results for breath analysis. Despite their advantages, PET film bags present several significant problems once the bag is filled:
(i) Permeability: PET film bags are permeable, especially to small molecules like water. This permeability can lead to the loss of volatile components and the ingress of contaminants, compromising the integrity of the sample.
(ii) Condensation: The issue of condensation within the bag is significant, particularly if its temperature is not controlled. Condensation can alter the concentration of VOCs in the sample, leading to inaccurate analysis results. This is particularly problematic when using techniques like SESI-HRMS, where humidity can affect the ionization process and the detection of certain compounds.
(iii) Sample Stability: Due to permeability and condensation issues, breath samples collected in PET film bags must be analyzed within a very short timeframe, typically less than two hours, to ensure reliable results. This requirement complicates the logistics of sample collection and analysis, especially in field settings or when samples need to be transported to a distant laboratory.
(iv) Fragility: PET film bags are relatively fragile, making them susceptible to damage during handling and transport. This fragility adds to the logistical challenges and increases the risk of sample loss or contamination.

In conclusion, there remains a need for an improved air storage unit that overcomes the limitations of currently available sampling solutions. The present invention contributes to these advancements by providing a robust air storage unit and associated methods that ensure the preservation and integrity of VOCs and other airborne substances from the point of collection to analysis. This innovation supports a wide range of applications, enhancing the ability to monitor, diagnose, and research air quality and its impacts effectively. In particular, this invention aims to provide a more reliable solution for breath analysis and other applications requiring precise air sample collection and preservation.

### SUMMARY OF THE INVENTION

. The present invention relates to an advanced air storage unit designed for the collection, preservation, and analysis of air samples, particularly volatile organic compounds (VOCs). The air storage unit comprises a film bag housed within a thermally insulated metal canister. This canister is equipped with an inlet valve, sample port, and secondary port, and optionally includes a heating system to maintain a consistent internal temperature.

The film bag, made from chemically inert materials such as Polyvinyl fluoride, Polyvinyl difluoride, and Polyimide, collapses when pressure is applied, ensuring minimal contamination and background gases. The rigid canister, constructed from materials like stainless steel and aluminum, provides a robust chemical barrier, significantly extending the integrity and storage duration of air samples by preventing the ingress of contaminants and the egress of sample molecules.

An innovative feature of the invention is its ability to preheat before sample collection, reducing the condensation of less volatile VOCs and thus preserving the sample's composition. For sample collection, the air storage unit can handle both high and low-pressure sources by using either direct inflow or an exhaust pump mechanism.

The invention also includes a preservation bed that maintains the temperature of the stored samples, reducing vapor condensation and ensuring sample integrity during transport. This system can be arranged in an array within a suitcase for convenient handling, shipping, and power management, ensuring continuous temperature control even during transit.

A notable application of this invention is in breath analysis, where the air storage unit can collect specific fractions of exhalations using a breath sampling inlet. This inlet ensures that only a predefined breath fraction is collected, enhancing the reproducibility and reliability of biomarker analysis in breath samples.

Overall, the invention provides a robust solution for long-term storage and analysis of air samples, with applications extending to various fields requiring precise and uncontaminated air sampling and analysis.

### BRIEF DESCRIPTION OF THE DRAWINGS

FIG. 1 shows a section view of an air storage unit of the present invention. This section view shows the sample port, the inlet valve, the film bag, the rigid canister, the insulation, the stopper, and the secondary port.

FIG. 2 shows a section view of the air storage unit of the present invention. This section view shows the sample port, the inlet valve, the film bag, the rigid canister, the insulation, the stopper, the internal heating system, and the electronics port.

FIG. 3 shows a front view of the air storage unit, where the rigid canister and the insulation are cut so that the film bag and the internal heating system are exposed. This view also shows the sample port, the inlet valve, the stoper, and the secondary port, and the electronics port.

FIG. 4 shows a front view of the air storage unit. This view shows the sample port, the inlet valve, the canister, the stoper, and the secondary port, and the electronics port.

FIG. 5 shows an air storage unit connected to a preservation bed, wherein the preservation bed provides a support to hold the air storage unit in place, and an electric cable to provide power and communication with the internal heating system.

FIG. 6 shows several air storage units sitting on their respective beds and stored in a suitcase.

FIG. 7 shows a block diagram of the controller of the suitcase and an array of preservation beds.

FIG. 8 shows a section view of the air storage unit, coupled with a breath sampling inlet. In addition to the air storage unit, this view shows the disposable mouthpiece with the antiviral filter, the main body with a channel that communicates the mouthpiece and the sample port, a second channel that communicates the mouthpiece with a first mouthpiece port, and an exhaust channel that connects with the secondary port.

FIG. 9 shows a section view of the air storage unit, coupled with a breath sampling inlet. In addition to the air storage unit, this view shows the disposable mouthpiece with the antiviral filter, the channel that communicates the mouthpiece and the sample port, a third channel that communicates the mouthpiece with a restriction followed by an exhaust and a second mouthpiece port, and two clamps that facilitate quick connection and disconnection of the air storage unit and the breath sampling inlet.

FIG. 10 shows a front view of the storage unit coupled with the breath sampling inlet. This view highlights the inlet valve, which is accessible and actionable with the breath sampling inlet in place, and the clamps.

FIG. 11 shows schematically the air storage unit coupled with the breath sampling inlet and a complete breath sampling system, which further incorporates a sensor module, an exhaust controller, tubes connecting the first and the second mouthpiece ports with the sensor module, and a tube connecting the exhaust channel with the exhaust controller. This figure also shows an individual exhaling into the sampling system, while looking at the visual indication of the sensor module.

FIG. 12 shows an embodiment of the air storage unit coupled with the breath sampling inlet and a complete breath sampling system, which further incorporates a sensor module, an exhaust valve, a holder to hold the sampling unit, tubes connecting the first and the second mouthpiece ports with the sensor module, and a tube connecting the exhaust channel with the exhaust valve. This figure also shows an individual exhaling into the sampling system, while looking at the display of the sensor module.

FIG. 13 shows a section view of an analyzer interface coupled with the air storage unit. This interface fluidly connects the sample port of the air storage unit with the sample inlet of the analyzer and a flow splitter that connects with the secondary port.

FIG. 14 shows another section view of the analyzer interface connected with the air storage unit. This view shows a detail of the flow splitter that receives a stream which pressurizes the rigid canister.

FIG. 15 shows the analyzer interface coupled with a Volatile Organic Compound ionizer and a mass spectrometer for analysis.

FIG. 16 shows the air storage unit coupled with the analyzer interface, a Volatile Organic Compound ionizer, and a mass spectrometer for analysis, wherein the content of the film bag is delivered to the ionizer and the analyzer.

FIG. 17 shows a detail view of the air storage unit coupled with the analyzer interface, showing the inlet tube of the analyzer, a tube that feeds the splitter, a small split gas exhaust and a large split gas exhaust, and the inlet valve.

### DETAILED DESCRIPTION OF THE INVENTION

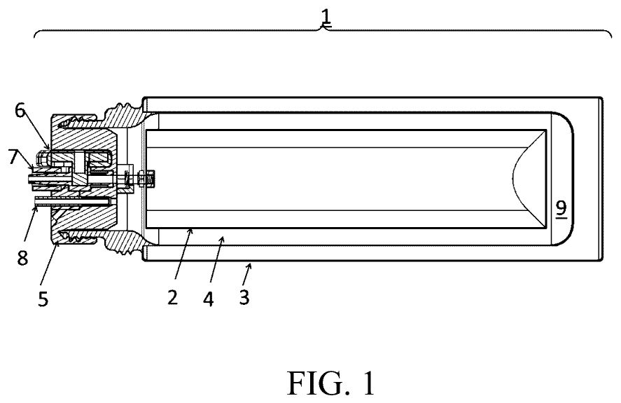

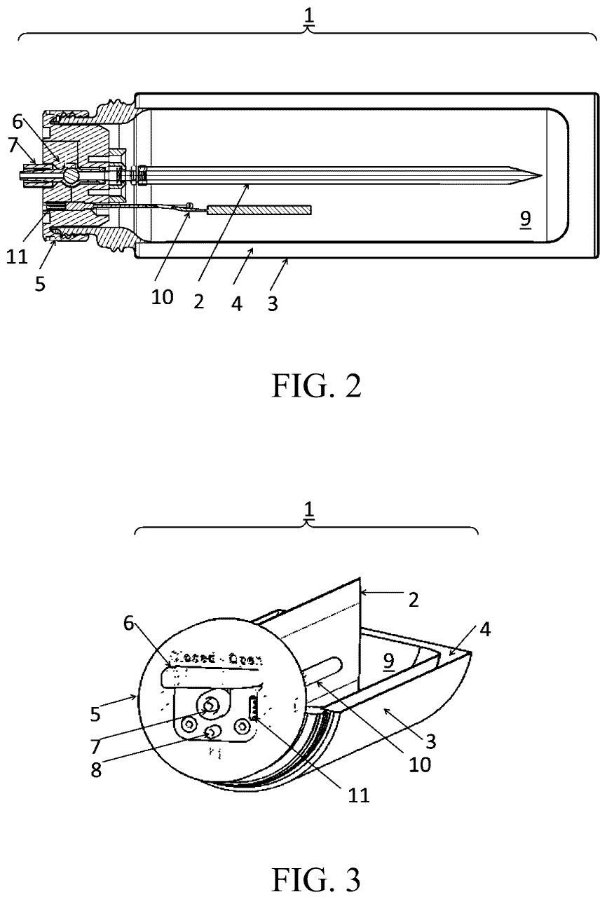

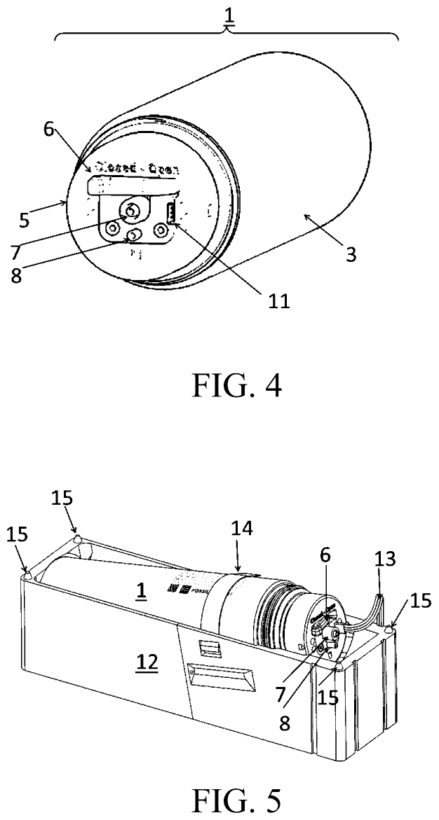

. FIG. 1 shows a section view of the air storage unit (1), which comprises a film bag (2) inside a metal canister (3) thermally insulated by an insulation layer (4). The metal canister is closed by a stopper (5) that houses an inlet valve (6) that communicates with a sample port (7) in the outer side of the stopper (5) and the film bag (2), a secondary port (8) that communicates the outer side of the stopper (5) with the region (9) inside the canister (3) and outside the film bag (2). Optionally, the air storage units further comprise a heating system (10) that is housed in the region (9) and is further electrically communicated with the outer side of the stopper though an electronics port (11), which are shown in FIGS. 2, 3 and 4.

The film bag is made of a chemically inert film material, such as Polyvinyl fluoride, Polyvinyl difluoride, Chlorotrifluoroethylene, Polyimide, Polyethylene terephthalate, other materials known to the skilled in the art, or a multilayer film. The rigid canister is be made of glass stainless steel, aluminum PEEK, or other rigid material with low permeability that provided a solid chemical barrier. The insulation can be achieved with insulating materials, including insulating gas and vacuum. The inlet valve is preferable made of an inert material.

To prepare an air storage unit, the film bag is chemically cleaned and emptied by applying pressure to the region (9) through the secondary port (8) with the inlet valve (6) is open so that the film bag collapses. Once the film bag is collapsed, the inlet valve is closed, and the pressure is released. The empty storage unit can be stored until used. Before it is used, the air storage unit is preheated by connecting the electronics port (11) to an external controller that provides power to the heating system (10) and logs the temperature inside the canister. Heating the air storage unit before collecting a sample prevents condensation of the less volatile Volatile Organic Compounds (VOCs) when a sample is collected.

Collecting a sample with the air storage unit when the pressure of the source of the sample is higher than the ambient pressure requires the steps of connecting the sample port to the sample, opening the inlet valve to allow the passage of the sample air into the bag film until the film bag is filled, and closing the inlet valve. As the sample air enters the film bag, the displaced gas of the region (9) exits through the secondary port. To control the flow of air that inflates the film bag, the secondary port of the air storage unit can be connected to an exhaust valve so that air is only sampled when the exhaust valve is opened.

Collecting a sample with the air storage unit when the pressure of the source of the sample is lower than the ambient pressure or is very low requires the steps of connecting the sample port to the sample, opening the inlet valve to allow the passage of the sample air into the bag film until the film bag is filled, and closing the inlet valve. To draw the sample air into the film bag, the secondary port is connected to an exhaust pump, or an exhaust valve further connected to an exhaust pump. When the exhaust valve or the pump are activated, the gas in the region (9) is sucked causing the film bag to draw sample air from the sample port.

Once the sample is stored in the bag film and the inlet valve is closed, the air storage unit is preserved in a preservation bed (12), as shown in FIG. 5. The preservation bed provides a support to hold the air storage unit in place, and an electric cable (13) to provide power and communication with the internal heating system. In this case, the preservation bed features a strap (14) to hold the air storage unit. The powered air storage unit maintains a constant temperature above ambient temperature, which helps reducing condensation of the vapors sampled and stored in the film bag. This helps maintaining the integrity of the samples for longer. In addition, the temperature inside the air storage unit can logged to assess and guarantee that the sample has been properly handled from collection to analysis.

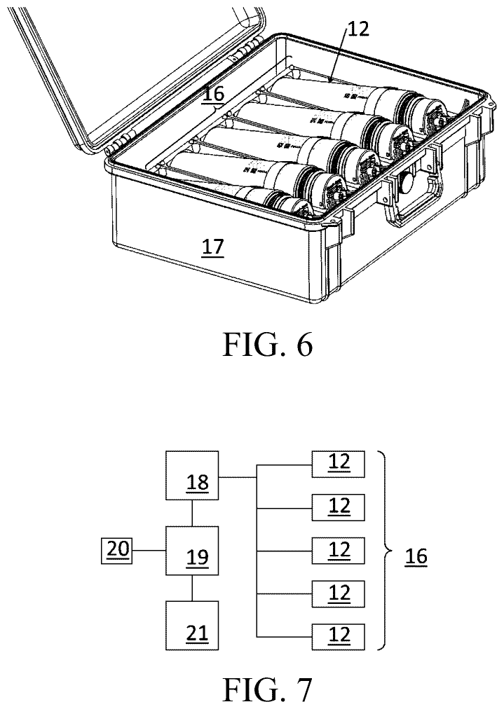

The preservation bed can feature socket joints (15) to facilitate piling several preservation beds together to form an array. FIG. 6 shows an array (16) of preservation beds (12) holding and connecting with their corresponding air storage units (1) in a suitcase (17). The suitcase incorporates an electronic controller and logging unit (18) that powers and logs the array (16), a power management unit (19) with a power inlet (20) and an optional battery (21). The corresponding diagram block of the electronics of the suitcase are illustrated in FIG. 7. The electronic controller comprises an interface that allows the user to define the temperature setpoint for the units in the array, an internal clock, means to log the temperature history of each unit in the array, and means to export this data. When external power is available, the power management unit uses external power to power the controller (18) and charges the battery if needed. When no external power is available, the power management unit suitcase uses battery to power the controller. This allows the temperatures to be always controlled, including when the suitcase is shipped from the sampling site to the analysis site.

Regular sampling bags have the advantages that they require a low pressure drop to collect the sample, and they are known (especially PET film bags) to hold low volatility molecules particularly well, but only for a few hours. The air storage unit of the present invention also has these advantages, but compared to regular sampling bags, the air storage unit has the following advantages:
(i) it retains low volatility molecules for longer because it is heated and the temperature is kept constant, this reduces condensation of molecules, especially for the larger VOCs.
(ii) it also retains small molecules for longer. Film bags are permeable to small molecules such as water. As an example, the humidity of a fresh two-liter breath sample with a 36° C. dew point reaches equilibrium with the ambient humidity in about two hours. In contrast, the material of the canister provides a non-permeable chemical barrier that maintains the integrity of sample for much longer.
(iii) the chemical barrier provided by the canister also precludes permeation of ambient contaminants into the sample. As a result, samples are a lot less susceptible to exposure to ambient contamination both before a sample is collected, and in the time between sample collection and analysis.
(iv) The film bag is fully emptied with a net pressure difference, so the amount of background gas potentially contributing to contamination is a lot lower. In addition, the gas used to pressurize the canister before use is ultra clean, such as clean N2, to reduce the likelihood of a contaminant diffusing into the bag film. This results in much lower background contamination.
(v) the likelihood of artifacts affecting the content of the samples during the time between sample collection and analysis due to inconsistent handling is much reduced. For instance, temperature variations during shipping can be quite substantial. This is avoided because the new system ensures a constant and traceable temperature. The mechanical integrity of the bags can also be affected during shipping, as transport handling can be very rough. Mechanical stress of the film bag are eliminated because the bag is protected inside the rigid canister.
(vi) the flow of sample gas entering the film bag can be controlled with a simple valve, and the valve does not introduce any contaminant because it is placed downstream of the storage unit and fluidly connected with the secondary port.

One important application of the present invention is for the collection and offline analysis of breath samples, where breath samples are collected in one site and analyzed elsewhere. The chemical composition of breath varies during the exhalation. For CO2, the shape of the CO2 concentration profile during the exhalation is known as capnography, and it is a subject of study that is used in intensive care and anesthesia among others. Similarly, other metabolites exhaled by the person have different profiles in the different parts of the exhalation. For instance, metabolites coming from the alveoli have a lower concentration in the first part of the exhalation and tend to rise and plateau at the end of the exhalation, whereas metabolites coming from the mouth show an initial spike followed by a lower signal as the exhalation washes mouth cavity. For this reason, in other to use the concentration of a metabolite in breath as a biomarker, said concentration level needs to be linked with the corresponding breath fraction. Obtaining the breath concentration of the different metabolites is easy when breath is analyzed in real time, but it is not possible when breath is collected, mixed in a container, and analyzed offline. To compensate for this, the present invention teaches a breath sampling inlet (22) device and a method to collect only a predefined fraction of the exhalation.

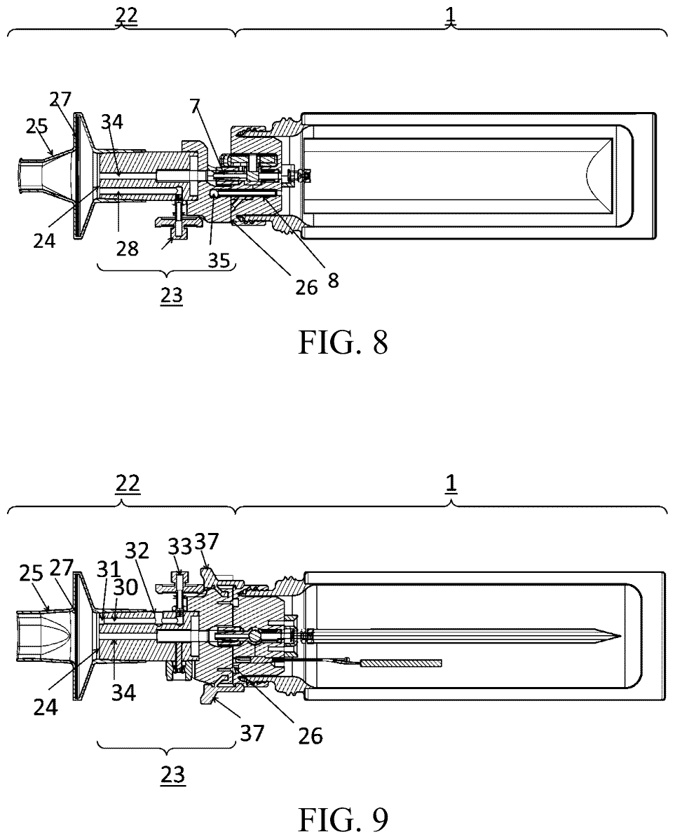

FIGS. 8 and 9 show two perpendicular section views of an air storage unit (1) coupled with a breath sampling inlet (22). The main body (23) of the breath sampling inlet (22) has a front side (24) that connects with a mouthpiece (25), and a back side (26) that connects with the air storage unit (1). In these figures, a disposable mouthpiece (25) with an antiviral filter (27) is shown for reference, but other mouthpieces and adaptors including but not excluding nose-space adaptors, face masks and other adaptors known to those skilled in the art could be used. The main body has a first channel (28) that communicates the mouthpiece with a first sensor port (29), a second channel (30) that communicates the mouthpiece with a restriction (31) followed by an exhaust (32) and a second sensor port (33), a main channel (34) that communicates the mouthpiece region and the sample port of the air storage unit, and an exhaust channel (35) that connects the secondary port (8) of the air storage unit (1) with and exhaust port (36).

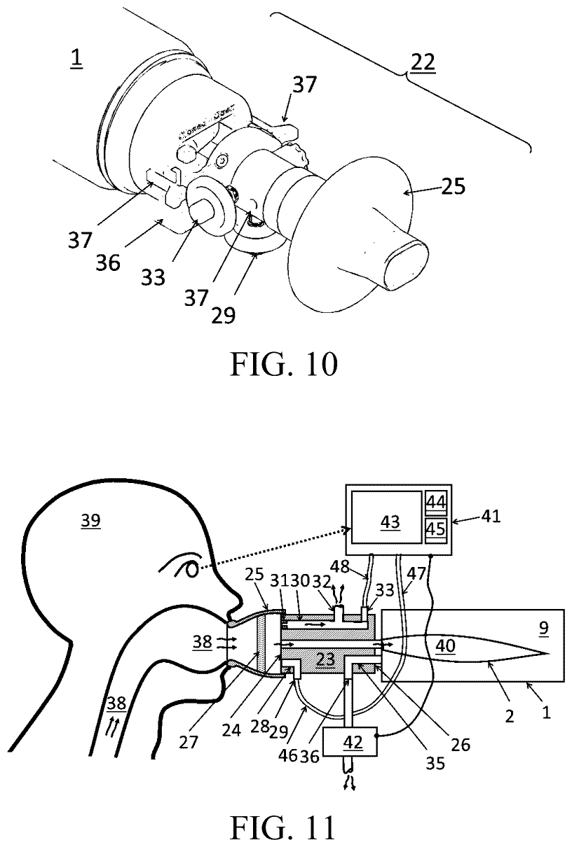

The breath sampling inlet (22) can have a clamping mechanism (37) to facilitates quick connection and disconnection of the breath sampling inlet (22) and the air storage unit (1), and a groove to facilitate access to the inlet valve (6). This arrangement, illustrated in FIG. 10, allows the user to collect several samples more conveniently.

In a more generic description embodiment of the present invention, the main body has a main channel (34) that communicates the front side (24) with the air storage unit (1), and at least one secondary channel (28) that communicates the front side (25) with at least one sensor port (29) and a secondary exhaust (11). This is so that the flow exhaled (38) by the person (39) to split in two so that the flow sampled (40) into the air storage unit (1) does not pass through the at least one channel connected to the sensor ports. This arrangement prevents any potential contaminants released by the sensors connected to the sensor ports from ever reaching the air storage unit (1). In addition, by making the main channel as straight and as short as possible, its exposed surface is minimized, and the flow is kept straight and laminar. This allows for the reduction of chemical contamination of the flow sampled (40) to the analyzer.

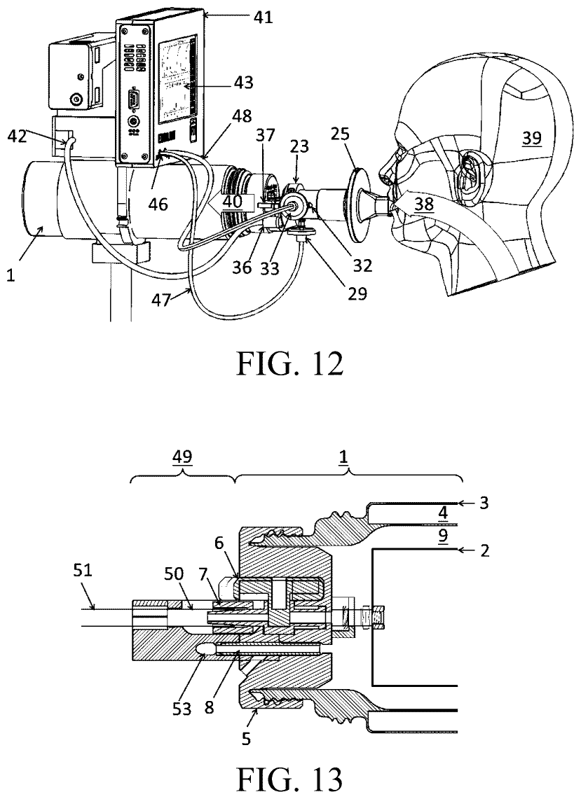

FIG. 11 illustrates schematically the air storage unit (1) coupled with a breath sampling inlet (22) further connected with a sensor module (41) and an exhaust controller (42), which can be a valve or a pump or a valve and a pump. FIG. 12 illustrates an embodiment of the breath sampling inlet (22) further connected with a sensor module (41) and an exhaust controller (42). The sensor module (41) connects with the sensor ports (29), and include a visual indication (43), a data processing unit (44) and a data storage unit (45). The visual indication (43) shows how strong the person is exhaling, and a visual target. The person (39) exhales while looking at the visual indication (43) and self-regulates how hard he/she is exhaling to match the target. The visual clue can be a moving element, a set of Light Emitting Diodes, it can be integrated in a screen, or other visual clues known to those skilled in the art. The sensor probes at least measure one of the following parameters: manometric pressure of the exhalation, exhaled flow rate, exhaled volume, exhaled CO2 concentration, exhaled humidity, absolute pressure.

The diameter of the second channel (30) and the exhaust (32) are defined so that the pressure drops along said channel (30) and said exhaust (32) is negligible in comparison with the pressure drop along said restriction (31), so that the flow rate (38) can be accurately calculated from the manometric pressure measurements of the pressure probe (46). The pressure probe (46) can be a pressure sensor or a tube connector communicating through a tube (47) with a pressure sensor integrated in the sensor module (41). In one embodiment of the present invention, this calculation is performed by the data processing unit (44), shown to the user as a visual indication (43) to show the evolution of the exhalation over time, and stored in the data storage unit (45). In another embodiment of the present invention, the exhaled flow data is used by the data processing (44) unit to determine the exhaled volume by numerically integrating flow over time. Optionally, the exhaled volume can be shown for the person to know how much has been exhaled.

In another embodiment of the present invention, a CO2 concentration probe measures the concentration of CO2 downstream of said calibrated restriction (31). The CO2 concentration probe can be a pressure sensor or a tube connector communicating through a second tube (48) with a pump and a CO2 sensor integrated in the sensor module (41). In one embodiment of the present invention, said second tube (48) can be made of a material permeable to humidity to prevent condensation of breath humidity in the pump or the CO2 sensor. Optionally, the pump and the CO2 sensors can be heated to prevent condensation of humidity. In one embodiment of the present invention, the CO2 concentration is processed by the data processing unit (44), shown to the user as an additional visual indication, and stored in the data storage unit (45). Similarly, another embodiment of the invention incorporates a humidity sensor probe. Another embodiment of the present invention comprises an absolute pressure sensor, which is used by the data processing unit to correct the measurements of other sensors caused by variations in absolute barometric pressure.

Using the measurements provided by the sensors and its internal algorithms, the sensor module (41) commands the exhaust controller (42) so that it allows the passage of gas when the person breathes out the predefined fraction of the exhalation. To collect a pre-defined fraction of the exhalation, in one embodiment of the present invention, the sensor module measures and/or computes the CO2 concentration or the flow rate, or the exhaled volume or a combination of them, commands the exhaust controller to collect the exhaled air when the CO2 concentration, the flow rate, the exhaled volume, or a combination of these signals meet a predefined Boolean combination of thresholds, and commands the exhaust controller to stop collecting when these signals reach a second Boolean combination of thresholds, or when the exhalation finishes. Several exhalations can be collected in one air storage unit with this method to collect a desired sample volume.

The chemical composition of breath varies within each exhalation, but it also varies from one exhalation to the next depending on several physiological and psychological factors. For instance, the energy expenditure of a person is different if he/she is moving, standing still, or sitting. This has an impact on the metabolism, the blood circulation, the breathing pattern, and ultimately the chemical composition of breath. Similarly, a psychological stressor can change the composition of breath. When working on the identification of biomarkers of diseases or medical treatment, this biological variability enters the datasets as a confounder, so it is important to eliminate such sources of biological variability in as much as possible. The present invention helps reduce these sources of biological variability in two ways: (i) by instructing all people exhaling though the breath inlet to exhale at the same flow rate with the aid of the visual indication, all exhalations are very similar. In addition, by asking all people to exhale the same volume at a fixed cadence, the breathing pattern of all people participating in a breath analysis study is equalized. A method to collect breath samples with the apparatus of the present invention comprises the steps of: instructing the person to (i) look at the visual indication, exhale through the mouthpiece and self-regulate the exhalation pressure to hit the flow rate target until a predefined exhaled volume is reached, wherein the visual indication provides information on exhaled flow rate and exhaled volume in real time. (ii) breath naturally for a predefined period of time, (iii) repeat steps ‘i’ and ‘ii’ for a predefined number of times N, (iv) detect the exhalations with the sensor module, (v) discard the first ‘N0’ exhalations, and (vi) collect the predefined fraction of the exhalations N0+1 until N. The exhalation fraction predefined by a set of thresholds. In particular, To collect a pre-defined fraction of the exhalation, the sensor module measures and/or computes the CO2 concentration or the flow rate, or the exhaled volume or a combination of them, commands the exhaust controller to collect the exhaled air when the CO2 concentration, the flow rate, the exhaled volume, or a combination of these signals meet a predefined Boolean combination of thresholds, and commands the exhaust controller to stop collecting when these signals reach a second Boolean combination of thresholds, or when the exhalation finishes.

When the person starts exhaling into the machine, his/her breathing pattern changes. As a result, the chemical composition of each consecutive exhalation changes. This can a problem because most data analysis algorithms require reproducible results. But the advantage is that, after breathing for a while following this controlled and guided breathing patter, the lungs reach a new equilibrium and the chemical composition of the consecutive exhalations become more reproducible [REF]. What is even more advantageous is that, while the natural breathing pattern of each person is different and unknown, the guided breathing pattern is known, reproducible, and repeatable across all people participating in a study. As a result, the apparatus and the method herein described greatly reduce biological variability linked to the breathing physiology.

Once the sample is stored, the inner valve is closed, and the air storage unit is preserved until it is brought to the analyzer and analyzed. To analyze the sample contained inside the film bag, the sample port is connected to the inlet of the analyzer. If the inlet of the analyzer has suction, opening the inlet valve will suffice to get the sample into the analyzer,

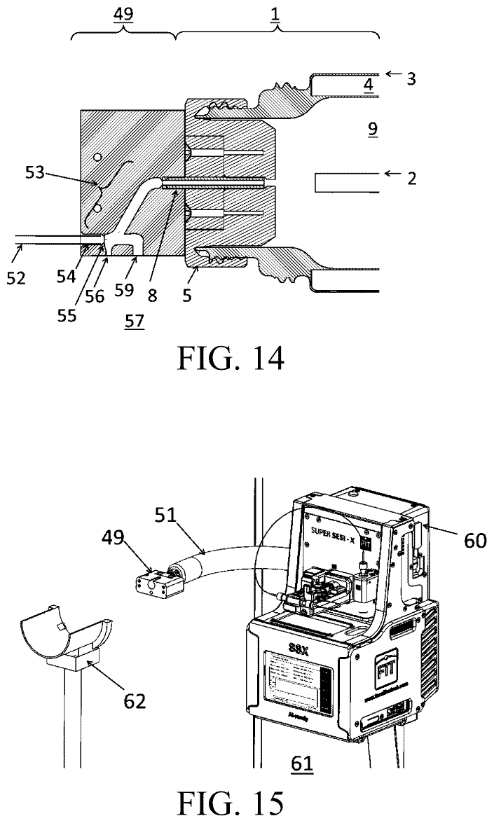

For analyzers that don't have suction power, such as the system described in Patent Application Number PCT/IB2017/057255 by the inventor of the present invention, pressure must be applied to the secondary port (8) to extract the sample air stored in the film bag (2). FIG. 13 and FIG. 14 show two section views of the air storage unit (1) coupled with an analyzer interface (49) the analyzer interface has a fluid conduct (50) that connects the sample port (7) with the inlet of the analyzer (51), a pressure gas inlet (52) that receives a flow of gas and passes it to an optional pressure splitter (53) consisting of a flow path having an inlet (54), an inlet restriction (55), an exhaust restriction (56) that communicates the pressure splitter with the room air (57), and a pressure channel (58) that communicates with the secondary port (8) of the air storage unit (1). When pressure is applied to the pressure gas inlet (52), the pressure splitter delivers a fraction of the pressure to the secondary port (8) so that the film bag is squeezed at the right analysis pressure. Optionally, the pressure splitter has a direct exhaust (59) that communicates the pressure splitter directly with the room air without restriction, so that the pressure in the pressure splitter can be rapidly switched from room pressure to analysis pressure simply by restricting the flow going through the direct exhaust (59) with a finger, a valve, or other similar means to stop the flow.

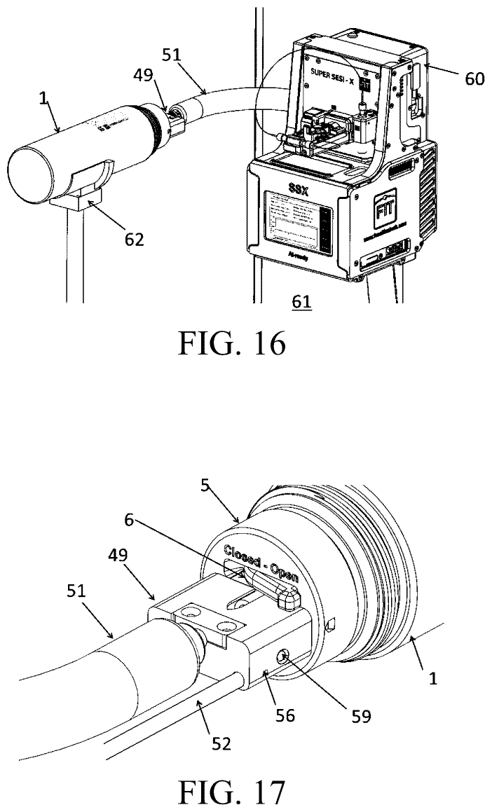

FIG. 15 shows the analyzer interface (49) coupled with a commercial ionization source (60), a mass spectrometer (61) used for the analysis of Volatile Organic Compounds and a holder (62) that serves to hold and position the air storage unit aligned with the analyzer interface (49) FIG. 16 shows the same arrangement with an air storage unit (1) coupled to it for analysis. The analyzer interface (49) is arranged to facilitate access to the inlet valve (6).

To analyze the content of an air storage unit (1), the air storage unit (1) is removed and disconnected from its preservation bed (12), connected to the analyzer interface (49), and the inlet valve (6) is opened. As a result, the pressure provided by the pressure splitter squeezes the film bag, and the sample is introduced in the analyzer. In one embodiment of the present invention, the analyzer comprises an ionizer, where vapors are ionized, and an ion analyzer, but other vapor and gas analyzers known to the skilled in the art can be used in combination with the present invention and are thus part of the present invention.

What is claimed is:

1. An air storage unit for the collection and preservation of airborne analytes in air samples, including volatile organic compounds (VOCs), gases, vapors, and aerosols, comprising: a chemically inert film bag; a rigid canister housing the film bag, the canister comprising: (i) an inlet valve; (ii) a sample port configured to deliver air into the film bag; (iii) a secondary port in communication with a region between the film bag and the canister wall; and a heating system configured to maintain a constant internal temperature during sample storage; wherein the film bag is collapsible under pressure applied through the secondary port, and expandable when the inlet valve is opened during sample collection.

2. The air storage unit of claim 1, wherein the film bag is made of a chemically inert material suitable for preserving airborne analytes, the material comprising polyvinyl fluoride (PVF), polyvinylidene fluoride (PVDF), chlorotrifluoroethylene polymer (CTFE), polyimide, polyethylene terephthalate (PET), multilayer films thereof, and functionally equivalent materials known to a person of ordinary skill in the art.

3. The air storage unit of claim 1, wherein the canister is made of a chemically inert, low-permeability material including stainless steel, aluminum, PEEK, PET, or functionally equivalent rigid materials.

4. The air storage unit of claim 1, wherein the heating system is configured for external power and control.

5. The air storage unit of claim 1, further comprising a temperature logging module configured to record and export a temperature history.

6. The air storage unit of claim 1, further comprising a preservation bed configured to support the air storage unit and maintain temperature, the bed optionally including a securing strap and an electrical connection to the heating system.

7. The air storage unit of claim 1, further comprising a flow control element coupled to the secondary port, the flow control element comprising a restrictor, a valve, a pump, or a combination thereof, or any functionally equivalent device configured to regulate internal pressure during sample collection or release.

8. A breath sampling system comprising: an air storage unit according to claim 1; and a breath sampling inlet operatively connected between a mouthpiece and the sample port of the air storage unit, the inlet comprising: (i) a sensor interface configured to detect one or more breath parameters selected from the group consisting of pressure, flow rate, carbon dioxide concentration, humidity, and volume; (ii) an exhaust controller operatively connected to the secondary port of the air storage unit; wherein the exhaust controller is configured to control the timing or volume of air collected based on sensor data.

9. The breath sampling system of claim 8, wherein the breath sampling inlet comprises: a main channel to the air storage unit; a secondary channel to the sensor interface; and an exhaust channel to the exhaust controller.

10. The breath sampling system of claim 8, wherein the breath sampling inlet comprises a connection mechanism configured for quick release and reconnection.

11. The breath sampling system of claim 8, wherein the sensor interface is configured to measure carbon dioxide concentration and flow rate, and includes a user interface for displaying a target exhalation profile.

12. The breath sampling system of claim 8, wherein the system is configured to collect a breath sample over multiple exhalations while excluding undesired portions of each exhalation.

13. The breath sampling system of claim 8, wherein the controller is configured to maintain the air storage unit at a predefined temperature and log temperature data.

14. The breath sampling system of claim 8, wherein the breath sampling inlet is configured to limit or divert breath flow away from the air storage unit via sensor channels.

15. A method for collecting and preserving a breath sample containing one or more airborne analytes, including volatile organic compounds (VOCs), gases, vapors, or aerosols, comprising: receiving at least one exhalation from a user; monitoring one or more breath parameters during the exhalation; detecting a predefined fraction of interest based on sensor signals; activating an exhaust controller to cause the fraction of interest to enter the film bag through the inlet valve; closing the inlet valve after the desired sample is collected; storing the air storage unit in a preservation bed; and optionally preheating the air storage unit before sample collection.

16. The method of claim 15, further comprising logging the exhaled volume, pressure, or flow rate during sampling and optionally storing data locally or externally.

17. The method of claim 15, wherein detecting a predefined breath fraction comprises evaluating a combination of threshold criteria based on sensor outputs.

18. The method of claim 15, wherein the user is guided by a user interface indicating a target exhalation flow rate or volume.

19. The method of claim 15, wherein the breath sample is analyzed using an ionization system for VOC analysis coupled to a mass spectrometer.

20. The method of claim 15, further comprising transporting the air storage unit in a portable housing comprising a battery-powered preservation system configured to maintain temperature.
